(19) United States
(12) Patent Application Publication
(10) Pub. No.: US 2024/0165184 A1
(43) Pub. Date: May 23, 2024

(54) Bee Pollen Patty

(71) Applicant: Villya LLC, Melbourne, FL (US)

(72) Inventor: William Miller, Melbourne, FL (US)

(21) Appl. No.: 18/518,946

(22) Filed: Nov. 24, 2023

Related U.S. Application Data
(60) Provisional application No. 63/384,819, filed on Nov. 23, 2022.

(51) Int. Cl. A61K 36/282; A61K 47/26; A61K 47/42; A61K 47/44

(52) U.S. Cl. CPC A61K 36/282; A61K 47/26; A61K 47/42; A61K 47/44

(57) ABSTRACT

A patty with Artemisia and a method for making the patty are described. Also, a nectar with Artemisia and a method for making the nectar are described. In some examples, the Artemisia may include Artemisia annua or Artemisia afra.

### RELATED APPLICATIONS

This application claims priority to U.S. Provisional Application No. 63/384,819, filed Nov. 23, 2022, whose contents are expressly incorporated herein by reference in their entirety for all purposes.

### TECHNICAL FIELD

Aspects of the disclosure relate generally to controlling parasites. More specifically, aspects of the disclosure relate to controlling parasites by providing hosts with one or more compounds that reduce or eliminate parasite populations.

### BACKGROUND

The Varroa destructor mite (also known as the Varroa destructor and the Varroa mite) is an external parasitic mite that attacks and feeds on various honey bees including the Apis cerana (the Asian honey bee) and Apis mellifera (the western honey bee). The disease caused by the mites is called varroosis. The Varroa mite can reproduce only in a honey bee colony. It attaches to the body of the bee and weakens the bee by sucking fat bodies. The Varroa destructor species is a vector for at least five debilitating bee viruses, including RNA viruses such as the deformed wing virus (DWV). A significant mite infestation often leads to the death of a honey bee colony, usually in the late autumn through early spring. The Varroa mite is the parasite with possibly the most pronounced economic impact on the beekeeping industry. Varroa is considered to be one of multiple stress factors contributing to the higher levels of bee losses around the world.

### SUMMARY

Aspects described herein may address these and other problems, and generally reduce issues associated with mite infestations in bee colonies. The following presents a simplified summary of various aspects described herein. This summary is not an extensive overview and is not intended to identify key or critical elements or to delineate the scope of the claims. The following summary merely presents some concepts in a simplified form as an introductory prelude to the more detailed description provided below.

Aspects described herein may allow for an improved composition of ingredients to help reduce or eliminate mite infestations in bee colonies. An artificial bee pollen patty (or cake) may be formulated with Artemisia as an anti-parasitical ingredient. In some aspects, the Artemisia is allowed to infuse into water and then filtered out. In other aspects, the Artemisia is combined into the patty with other ingredients.

These features, along with many others, are discussed in greater detail below.

### BRIEF DESCRIPTION OF THE DRAWINGS

The present disclosure is illustrated by way of example and not limited to the accompanying figures in which like reference numerals indicate similar elements and in which:

FIG. 1 depicts an example of a bee pollen cake formed from various examples;

FIG. 2 is an example of a method for preparing a pollen cake;

FIG. 3 is an example of a method for preparing a pollen cake;

FIG. 4 depicts an example of a container with artificial nectar from various examples;

FIG. 5 is an example of a method for preparing nectar; and

FIG. 6 is an example of a method for preparing nectar.

### DETAILED DESCRIPTION

Various aspects relate to the creation of an artificial bee pollen patty with sugars and Artemisia (commonly known as wormwood). As described herein, two species of Artemisia are described herein (Artemisia annua and Artemisia afra). Other species of Artemisia having anti-parasitic compounds may be used in addition to Artemisia annua and/or Artemisia afra or in lieu of Artemisia annua and Artemisia afra. Other aspects relate to creating an artificial nectar with Artemisia annua and/or Artemisia afra. The difference in weight between bees and parasitic mites may be used to adjust the concentration of Artemisia in an artificial pollen patty and/or in the artificial nectar. For instance, if Artemisia is toxic to bees at a concentration greater than or equal to 88 mg/kg and a bee weighs, on average, 0.11 g to 0.2 g, then bee toxicity would range from 0.00968 mg to 0.0176 mg. Keeping the concentration of Artemisia between 10 to 20 mg/kg (one-eighth to one-quarter of bee toxicity) is desired.

As described here, a patty or cake is designed to rid the colony of mites in the brood cells. The patty is made available outside a hive for worker bees. The worker bees feed on the patty (full of protein and sugar and infused with Artemisia) and in turn, feed the bee larva by filling the brood cell with this food. As such, the patty is designed to address the issue of mites in the brood cell.

Also, another embodiment relates to a nectar comprised of one or more sugars infused with Artemisia. The nectar is intended to be ingested by workers, drones, and queen bees as food to kill the Varroa mite that is attached to them. The nectar-related embodiment may be beneficial in preventing the mites from attaching to them. The nectar is specifically designed to treat the mature bees in the colony.

In the following description of the various embodiments, reference is made to the accompanying drawings, which form a part hereof, and which are shown by way of illustration of various embodiments in which aspects of the disclosure may be practiced. It is to be understood that other embodiments may be utilized and structural and functional modifications may be made without departing from the scope of the present disclosure. Aspects of the disclosure are capable of other embodiments and of being practiced or being carried out in various ways. Also, it is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed thereafter and equivalents thereof as well as additional items and equivalents thereof.

By way of introduction, aspects discussed herein may relate to methods and techniques for providing an artificial bee pollen patty and/or nectar with sugars and Artemisia. The sugar may be used to attract bees. The Artemisia is ingested by the bee and functions as a miticide to eradicate the mites that may be on or in the bee and/or in the bee colony. The consumption of the Artemisia may provide three antiparasitic benefits to bees.

A first benefit may include direct coating of its mouth and digestive components with Artemisia. To the extent that any mites are in or around the mouth of the bees, the Artemisia may be concentrated in those areas and increase the exposure of mites to the Artemisia.

A second benefit may include the ingestion of the Artemisia and eventual storage in the bee's fat cells. To the extent that the mites attempt to feed off the bee's fat cells, the mites would have exposure to the Artemisia at a higher concentration than that experienced by the bee. If the Artemisia is in the fat cells, the Artemisia may prevent the mite from attaching at all.

A third benefit may include how bees feed bee larvae. As adult bees feed bee larvae by regurgitating food, the food may be mixed with Artemisia and fed directly to the larva. Mites may be capped in the brood comb with the bee larva, attach to, and feed off the bee larva. To the extent bees cap the brood comb with beeswax, the beeswax may contain residual amounts of Artemisia as well as any other material used to form or line the cocoons or food placed within the cocoons. Allowing sufficient Artemisia to be placed into the brood comb may kill the mites or prevent the mites from attaching to the bee larva. As such, any mites present in the hive may be controlled by residual Artemisia from outside the hive.

Further, there is also a possibility that the Artemisia may modulate the bee's pheromones to the extent that they would be less of an attractant to mites.

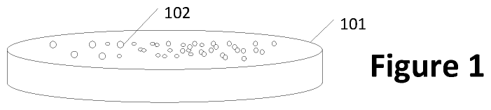

FIG. 1 illustrates an example of an artificial bee pollen patty 101. The pollen patty 101 may be formed from a mixture described below and allowed to dry. The pollen patty may contain, for example, small particles of Artemisia 102 in the pollen patty.

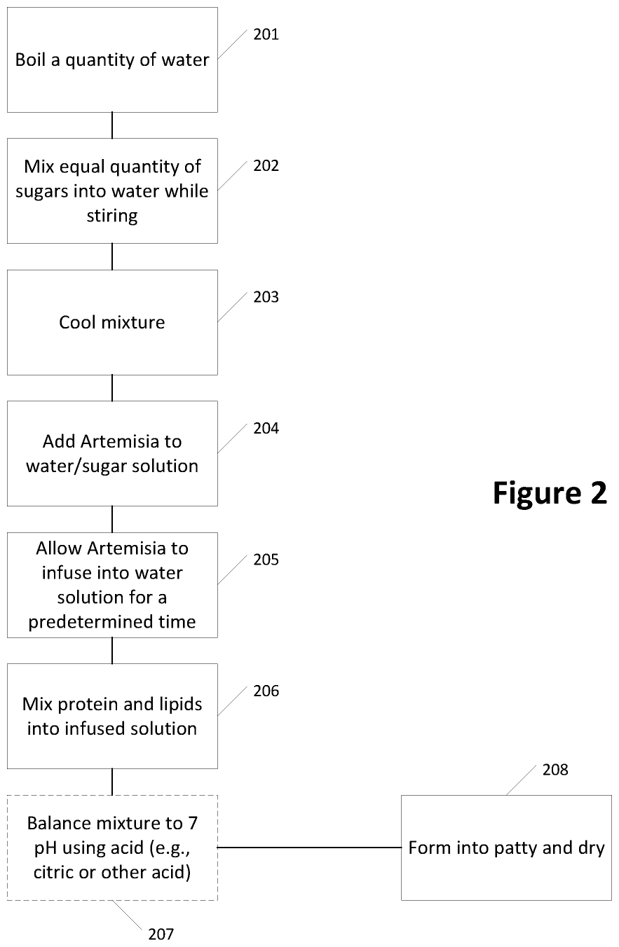

FIG. 2 shows a process for forming an artificial bee pollen patty.

A process for forming the artificial bee pollen patty 101 may include the steps shown in FIG. 2. In step 201, a quantity of water may be brought to a boil. In step 202, an equal amount of sugars (described below) is added to the hot water and stirred to allow the sugars to dissolve. In effect, a simple syrup is made from one or more sugars (sucrose, fructose, and/or glucose) and water.

In step 203, the mixture is allowed to cool. Once this simple syrup is made the Artemisia is allowed to soak and infuse the syrup. The percentage of Artemisia is adjusted for the purpose of killing the Varroa mite but protecting the honey bee from toxicity. The percentage of Artemisia may be adjusted in the final product from 0.025% to 40% as needed. The anatomy of the bee will allow the absorption of the sugar molecules during normal digestion. Similarly, the antiviral properties of Artemisia may further protect bees from viral infections brought into a hive via Varroa mites. The Varroa mites find the bees through the detection of the bees' pheromones. There is an opportunity to further prevent mites from attaching to bees and/or entering a hive by masking or disrupting the mites' sensory attraction to bees.

In step 204, the Artemisia is added to the water/sugar solution. The Artemisia added in step 204 may include leaves and/or twigs in their natural state or having been ground into a powder to increase their surface area. In step 205, the Artemisia is allowed to infuse into the water over a period of time (e.g., 24-48 hours or longer). In step 206, protein and lipids may be added to the infused solution. In step 207, the pH of the mixture may be checked and adjusted with an acid (e.g., citric acid or other acid) to around 7 pH (e.g., within a range of 6.0 pH to 7.5 pH or 6.9 pH to 7.1 pH, for instance). In step 208, the compound is shaped into a patty and allowed to dry.

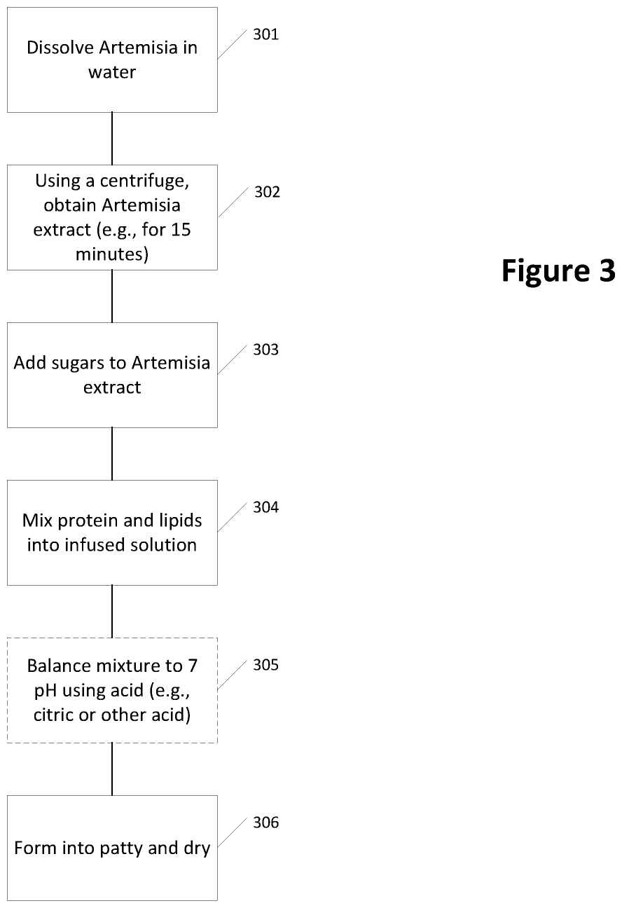

FIG. 3 shows a method for forming a pollen patty. In step 301, Artemisia is dissolved (or at least moistened) in water. The Artemisia added to the water in step 301 may include leaves and/or twigs in their natural state or having been ground into a powder to increase their surface area. In step 302, a centrifuge may be used to obtain an extract of Artemisia. In one example, the centrifuge may be run for approximately 15 minutes to extract the liquids from the solids. In step 303, a simple syrup of the sugars is added to the Artemisia extract. In step 304, proteins and lipids are added to the infused solution. If needed, the mixture may be tested and its pH adjusted in step 305 to 7 using an acid (e.g., citric acid or other acid) to approximately 7 pH (e.g., within a range of 6.0 pH to 7.5 pH or 6.9 pH to 7.1 pH, for instance). In step 306, the mixture may be formed into a patty and allowed to dry.

In FIGS. 2 and 3, the Artemisia solution may be used unfiltered. Alternatively, the Artemisia solution may be strained and filtered before being mixed with the protein and lipids.

One example of the compound for the pollen patty may include 50% Pea Protein; 3% Coconut oil; 37% (Fructose) or 50/50% a mix of (Fructose) and Table Sugar (sucrose); and 1-40% Artemisia afra or annua. Other examples are shown below in the following tables. The protein may include pea protein or similar protein. The lipids may include coconut oil or similar lipids. The volume measurements for the sugars are referenced in their simple syrup form (and designated with an asterisk “*”):

TABLE 1

| | Amount in tablespoons | Percent by volume |
|---|---|---|
| Protein (e.g., pea protein) | 5.25 | 33.38% |
| Fructose solution* | 6.00 | 38.14% |
| Sucrose solution* | 2.00 | 12.71% |
| Lipid (e.g., coconut oil) | 2.00 | 12.71% |
| Artemisia Afra or Annua | 0.48 | 3.05% |
| Total | 15.73 | 100.00% |

TABLE 2

| | Amount in tablespoons | Percent by volume |
|---|---|---|
| Protein (e.g., pea protein) | 5.25 | 32.71% |
| Fructose solution* | 6.00 | 37.38% |
| Sucrose solution* | 2.00 | 12.46% |
| Lipid (e.g., coconut oil) | 2.00 | 12.46% |
| Artemisia Afra or Annua | 0.80 | 4.98% |
| Total | 16.05 | 100.00% |

TABLE 3

| | Amount in tablespoons | Percent by volume |
|---|---|---|
| Protein (e.g., pea protein) | 5.25 | 31.16% |
| Fructose solution* | 6.00 | 35.61% |
| Sucrose solution* | 2.00 | 11.87% |
| Lipid (e.g., coconut oil) | 2.00 | 11.87% |
| Artemisia Afra or Annua | 1.60 | 9.50% |
| Total | 16.85 | 100.00% |

TABLE 4

| | Amount in tablespoons | Percent by volume |
|---|---|---|
| Protein (e.g., pea protein) | 5.25 | 30.97% |
| Fructose solution* | 6.00 | 35.40% |
| Sucrose solution* | 2.00 | 11.80% |
| Lipid (e.g., coconut oil) | 2.00 | 11.80% |
| Artemisia Afra or Annua | 1.70 | 10.03% |
| Total | 16.95 | 100.00% |

TABLE 5

| | Amount in tablespoons | Percent by volume |
|---|---|---|
| Protein (e.g., pea protein) | 5.25 | 28.46% |
| Fructose solution* | 6.00 | 32.52% |
| Sucrose solution* | 2.00 | 10.84% |
| Lipid (e.g., coconut oil) | 2.00 | 10.84% |
| Artemisia Afra or Annua | 3.20 | 17.34% |
| Total | 18.45 | 100.00% |

TABLE 6

| | Amount in tablespoons | Percent by volume |
|---|---|---|
| Protein (e.g., pea protein) | 5.25 | 27.56% |
| Fructose solution* | 6.00 | 31.50% |
| Sucrose solution* | 2.00 | 10.50% |
| Lipid (e.g., coconut oil) | 2.00 | 10.50% |
| Artemisia Afra or Annua | 3.80 | 19.95% |
| Total | 19.05 | 100.00% |

TABLE 7

| | Amount in tablespoons | Percent by volume |
|---|---|---|
| Protein (e.g., pea protein) | 50.00 | 54.95% |
| Fructose solution* | 37.00 | 40.66% |
| Sucrose solution* | 0.00 | 0.00% |
| Lipid (e.g., coconut oil) | 3.00 | 3.30% |
| Artemisia Afra or Annua | 1.00 | 1.10% |
| Total | 91 | 100.00% |

TABLE 8

| | Amount in tablespoons | Percent by volume |
|---|---|---|
| Protein (e.g., pea protein) | 50.00 | 38.46% |
| Fructose solution* | 37.00 | 28.46% |
| Sucrose solution* | 0.00 | 0.00% |
| Lipid (e.g., coconut oil) | 3.00 | 2.31% |
| Artemisia Afra or Annua | 40.00 | 30.77% |
| Total | 130 | 100.00% |

TABLE 9

| | Amount in tablespoons | Percent by volume |
|---|---|---|
| Protein (e.g., pea protein) | 50.00 | 33.33% |
| Fructose solution* | 37.00 | 24.67% |
| Sucrose solution* | 0.00 | 0.00% |
| Lipid (e.g., coconut oil) | 3.00 | 2.00% |
| Artemisia Afra or Annua | 60.00 | 40.00% |
| Total | 150 | 100.00% |

TABLE 10

| | Amount in tablespoons | Percent by volume |
|---|---|---|
| Protein (e.g., pea protein) | 50.00 | 54.95% |
| Fructose solution* | 18.50 | 20.33% |
| Sucrose solution* | 18.50 | 20.33% |
| Lipid (e.g., coconut oil) | 3.00 | 3.30% |
| Artemisia Afra or Annua | 1.00 | 1.10% |
| Total | 91 | 100.00% |

TABLE 11

| | Amount in tablespoons | Percent by volume |
|---|---|---|
| Protein (e.g., pea protein) | 50.00 | 38.46% |
| Fructose solution* | 18.50 | 14.23% |
| Sucrose solution* | 18.50 | 14.23% |
| Lipid (e.g., coconut oil) | 3.00 | 2.31% |
| Artemisia Afra or Annua | 40.00 | 30.77% |
| Total | 130 | 100.00% |

TABLE 12

| | Amount in tablespoons | Percent by volume |
|---|---|---|
| Protein (e.g., pea protein) | 50.00 | 33.33% |
| Fructose solution* | 18.50 | 12.33% |
| Sucrose solution* | 18.50 | 12.33% |
| Lipid (e.g., coconut oil) | 3.00 | 2.00% |
| Artemisia Afra or Annua | 60.00 | 40.00% |
| Total | 150 | 100.00% |

The following tables identify additional ratios of components as well as whether ants were attracted to the patty. For reference, each pollen cake is approximately 0.25 oz.:

TABLE 13

| | Amount in | Percent by | | | Amount |
|---|---|---|---|---|---|
| Pollen Cake | tablespoons | volume | Process | Ants | used |
| Protein (e.g., pea | 5.25 | 33.38% | | | |
| protein) | | | | | |
| Fructose solution* | 6.00 | 38.14% | | | |
| Sucrose solution* | 2.00 | 12.71% | | | |
| Lipid (e.g., coconut | 2.00 | 12.71% | | | |
| oil) | | | | | |
| Artemisia Annua | 0.48 | 3.05% | | | |
| Total | 15.73 | 100.00% | Strained | Yes | 14 |
| | | | Filtered | | TBSP |

TABLE 14

| | Amount in | Percent by | | | Amount |
|---|---|---|---|---|---|
| Pollen Cake | tablespoons | volume | Process | Ants | used |
| Protein (e.g., pea | 7.50 | 40.98% | | | |
| protein) | | | | | |
| Fructose solution* | 6.00 | 32.79% | | | |
| Sucrose solution* | 2.00 | 10.93% | | | |
| Lipid (e.g., coconut | 2.00 | 10.93% | | | |
| oil) | | | | | |
| Artemisia Annua | 0.80 | 4.37% | | | |
| Total | 18.3 | 100.00% | Strained | Yes | 12 |
| | | | Filtered | | TBSP |

TABLE 15

| | Amount in | Percent by | | | Amount |
|---|---|---|---|---|---|
| Pollen Cake | tablespoons | volume | Process | Ants | used |
| Protein (e.g., pea | 7.50 | 39.27% | | | |
| protein) | | | | | |
| Fructose solution* | 6.00 | 31.41% | | | |
| Sucrose solution* | 2.00 | 10.47% | | | |
| Lipid (e.g., coconut | 2.00 | 10.47% | | | |
| oil) | | | | | |
| Artemisia Annua | 1.60 | 8.38% | | | |
| Total | 19.1 | 100.00% | Strained | Few | 12 |
| | | | Filtered | | TBSP |

TABLE 16

| | Amount in | Percent by | | | Amount |
|---|---|---|---|---|---|
| Pollen Cake | tablespoons | volume | Process | Ants | used |
| Protein (e.g., pea | 7.50 | 36.23% | | | |
| protein) | | | | | |
| Fructose solution* | 6.00 | 28.99% | | | |
| Sucrose solution* | 2.00 | 9.66% | | | |
| Lipid (e.g., coconut | 2.00 | 9.66% | | | |
| oil) | | | | | |
| Artemisia Annua | 3.20 | 15.46% | | | |
| Total | 20.7 | 100.00% | Strained | No | 12 |
| | | | Filtered | | TBSP |

TABLE 17

| | Amount in | Percent by | | | Amount |
|---|---|---|---|---|---|
| Pollen Cake | tablespoons | volume | Process | Ants | used |
| Protein (e.g., pea | 7.50 | 42.47% | | | |
| protein) | | | | | |
| Fructose solution* | 6.00 | 33.98% | | | |
| Sucrose solution* | 2.00 | 11.33% | | | |
| Lipid (e.g., coconut | 2.00 | 11.33% | | | |
| oil) | | | | | |
| Artemisia Annua | 0.16 | 0.91% | | | |
| Total | 17.66 | 100.00% | Strained | Yes | 12 |
| | | | Filtered | | TBSP |

TABLE 18

| | Amount in | Percent by | | | Amount |
|---|---|---|---|---|---|
| Pollen Cake | tablespoons | volume | Process | Ants | used |
| Protein (e.g., pea | 7.50 | 41.71% | | | |
| protein) | | | | | |
| Fructose solution* | 6.00 | 33.37% | | | |
| Sucrose solution* | 2.00 | 11.12% | | | |
| Lipid (e.g., coconut | 2.00 | 11.12% | | | |
| oil) | | | | | |
| Artemisia Afra | 0.48 | 2.67% | | | |
| Total | 17.98 | 100.00% | Strained | Yes | 12 |
| | | | Filtered | | TBSP |

TABLE 19

| | Amount in | Percent by | | | Amount |
|---|---|---|---|---|---|
| Pollen Cake | tablespoons | volume | Process | Ants | used |
| Protein (e.g., pea | 7.50 | 40.98% | | | |
| protein) | | | | | |
| Fructose solution* | 6.00 | 32.79% | | | |
| Sucrose solution* | 2.00 | 10.93% | | | |
| Lipid (e.g., coconut | 2.00 | 10.93% | | | |
| oil) | | | | | |
| Artemisia Afra | 0.80 | 4.37% | | | |
| Total | 18.3 | 100.00% | Strained | Yes | 12 |
| | | | Filtered | | TBSP |

TABLE 20

| | Amount in | Percent by | | | Amount |
|---|---|---|---|---|---|
| Pollen Cake | tablespoons | volume | Process | Ants | used |
| Protein (e.g., pea | 7.50 | 39.27% | | | |
| protein) | | | | | |
| Fructose solution* | 6.00 | 31.41% | | | |
| Sucrose solution* | 2.00 | 10.47% | | | |
| Lipid (e.g., coconut | 2.00 | 10.47% | | | |
| oil) | | | | | |
| Artemisia Afra | 1.60 | 8.38% | | | |
| Total | 19.1 | 100.00% | Strained | No | 12 |
| | | | Filtered | | TBSP |

TABLE 21

| | Amount in | Percent by | | | Amount |
|---|---|---|---|---|---|
| Pollen Cake | tablespoons | volume | Process | Ants | used |
| Protein (e.g., pea | 7.50 | 36.23% | | | |
| protein) | | | | | |
| Fructose solution* | 6.00 | 28.99% | | | |
| Sucrose solution* | 2.00 | 9.66% | | | |
| Lipid (e.g., coconut | 2.00 | 9.66% | | | |
| oil) | | | | | |
| Artemisia Afra | 3.20 | 15.46% | | | |
| Total | 20.7 | 100.00% | Strained | No | 12 |
| | | | Filtered | | TBSP |

TABLE 22

| | Amount in | Percent by | | | Amount |
|---|---|---|---|---|---|
| Pollen Cake | tablespoons | volume | Process | Ants | used |
| Protein (e.g., pea | 7.50 | 42.47% | | | |
| protein) | | | | | |
| Fructose solution* | 6.00 | 33.98% | | | |
| Sucrose solution* | 2.00 | 11.33% | | | |
| Lipid (e.g., coconut | 2.00 | 11.33% | | | |
| oil) | | | | | |
| Artemisia Afra | 0.16 | 0.91% | | | |
| Total | 17.66 | 100.00% | Strained | Yes | 12 |
| | | | Filtered | | TBSP |

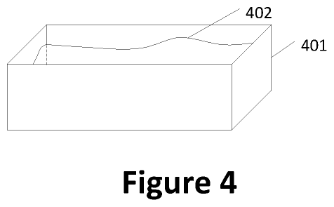

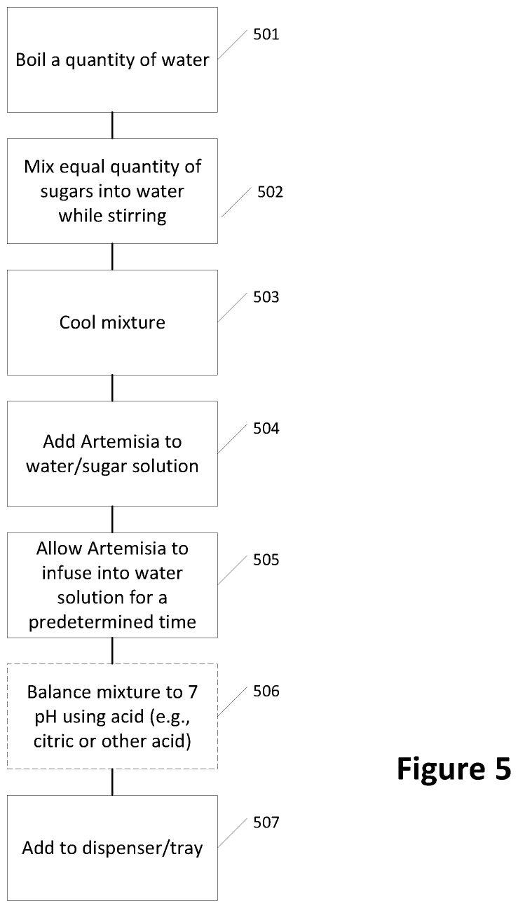

FIG. 4 shows an example of a container 401 with a quantity of nectar 402. FIG. 5 shows an example process of making the nectar. In step 501, a quantity of water is boiled. In step 502, an equal quantity of a sugar or combination of sugars is added to the water while stirring. The sugar or sugars and water form a simple syrup. In step 503, the mixture is cooled and Artemisia is added to the water/sugar solution in step 504. In step 505, the Artemisia is allowed to infuse into the water for a predetermined period of time (e.g., 24-48 hours or longer). In step 506, if needed, the mixture may be tested and its pH adjusted to 7 using an acid (e.g., citric acid or other acid) to approximately 7 pH (e.g., within a range of 6.0 pH to 7.5 pH or 6.9 pH to 7.1 pH, for instance).

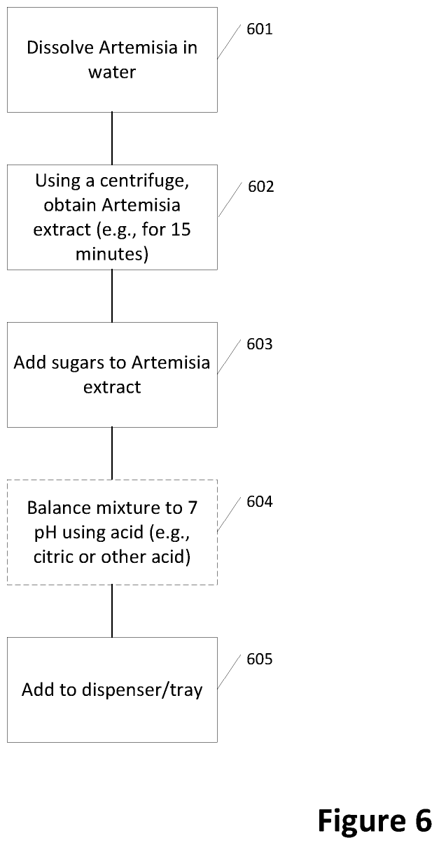

FIG. 6 shows a method for creating nectar. In step 601, Artemisia is dissolved (or at least moistened) in water. The Artemisia added to the water in step 601 may include leaves and/or twigs in their natural state or having been ground into a powder to increase their surface area. In step 602, a centrifuge may be used to obtain an extract of Artemisia. In one example, the centrifuge may be run for approximately 15 minutes to extract the liquids from the solids. In step 603, a simple syrup of the sugars is added to the Artemisia extract. If needed, the mixture may be tested and its pH adjusted in step 604 using an acid (e.g., citric acid or other acid) to approximately 7 pH (e.g., within a range of 6.0 pH to 7.5 pH or 6.9 pH to 7.1 pH, for instance). In step 605, the mixture may be added to a dispenser or tray.

In FIGS. 5 and 6, the Artemisia solution may be used unfiltered. Alternatively, the Artemisia solution may be strained and filtered before being added to a dispenser/tray.

The following tables are examples of the ratio of compounds for the nectar.

TABLE 23

| | Amount in teaspoons | Percent by volume |
|---|---|---|
| Fructose solution* | 1.00 | 48.54% |
| Sucrose solution* | 0.50 | 24.27% |
| Glucose solution* | 0.50 | 24.27% |
| Artemisia Afra or Annua | 0.06 | 2.91% |
| Total | 2.06 | 100.00% |

TABLE 24

| | Amount in teaspoons | Percent by volume |
|---|---|---|
| Fructose solution* | 1.50 | 72.82% |
| Sucrose solution* | 0.25 | 12.14% |
| Glucose solution* | 0.25 | 12.14% |
| Artemisia Afra or Annua | 0.06 | 2.91% |
| Total | 2.06 | 100.00% |

TABLE 25

| | Amount in teaspoons | Percent by volume |
|---|---|---|
| Fructose solution* | 1.00 | 47.62% |
| Sucrose solution* | 0.50 | 23.81% |
| Glucose solution* | 0.50 | 23.81% |
| Artemisia Afra or Annua | 0.10 | 4.76% |
| Total | 2.1 | 100.00% |

TABLE 26

| | Amount in teaspoons | Percent by volume |
|---|---|---|
| Fructose solution* | 1.00 | 45.45% |
| Sucrose solution* | 0.50 | 22.73% |
| Glucose solution* | 0.50 | 22.73% |
| Artemisia Afra or Annua | 0.20 | 9.09% |
| Total | 2.2 | 100.00% |

TABLE 27

| | Amount in teaspoons | Percent by volume |
|---|---|---|
| Fructose solution* | 1.00 | 45.05% |
| Sucrose solution* | 0.50 | 22.52% |
| Glucose solution* | 0.50 | 22.52% |
| Artemisia Afra or Annua | 0.22 | 9.91% |
| Total | 2.22 | 100.00% |

TABLE 28

| | Amount in teaspoons | Percent by volume |
|---|---|---|
| Fructose solution* | 1.00 | 41.67% |
| Sucrose solution* | 0.50 | 20.83% |
| Glucose solution* | 0.50 | 20.83% |
| Artemisia Afra or Annua | 0.40 | 16.67% |
| Total | 2.4 | 100.00% |

TABLE 29

| | Amount in | Percent by |
|---|---|---|
| | teaspoons | volume |
| Fructose solution | 1.00 | 40.00% |
| Sucrose solution | 0.50 | 20.00% |
| Glucose solution* | 0.50 | 20.00% |
| Artemisia Afra or Annua | 0.50 | 20.00% |
| Total | 2.5 | 100.00% |

TABLE 30

| | | | Amount used | |
|---|---|---|---|---|
| | Amount in | Percent by | (5x initial | |
| Nectar | teaspoons | volume | ratio) | Process |
| Fructose | 1.00 | 48.54% | 5.00 | |
| solution* | | | | |
| Sucrose | 0.50 | 24.27% | 2.50 | |
| solution* | | | | |
| Glucose | 0.50 | 24.27% | 2.50 | |
| solution* | | | | |
| Artemisia Annua | 0.06 | 2.91% | 0.30 | |
| Total | 2.06 | 100.00% | | Strained |
| | | | | Filtered |

TABLE 31

| | | | Amount used | |
|---|---|---|---|---|
| | Amount in | Percent by | (5x initial | |
| Nectar | teaspoons | volume | ratio) | Process |
| Fructose | 1.00 | 47.62% | 5.00 | |
| solution* | | | | |
| Sucrose | 0.50 | 23.81% | 2.50 | |
| solution* | | | | |
| Glucose | 0.50 | 23.81% | 2.50 | |
| solution' | | | | |
| Artemisia Annua | 0.10 | 4.76% | 0.50 | |
| Total | 2.10 | 100.00% | | Strained |
| | | | | Filtered |

TABLE 32

| | | | Amount used | |
|---|---|---|---|---|
| | Amount in | Percent by | (5x initial | |
| Nectar | teaspoons | volume | ratio) | Process |
| Fructose | 1.00 | 45.45% | 5.00 | |
| solution* | | | | |
| Sucrose | 0.50 | 22.73% | 2.50 | |
| solution* | | | | |
| Glucose | 0.50 | 22.73% | 2.50 | |
| solution™ | | | | |
| Artemisia Annua | 0.20 | 9.09% | 1.00 | |
| Total | 2.20 | 100.00% | | Strained |
| | | | | Filtered |

TABLE 33

| | | | Amount used | |
|---|---|---|---|---|
| | Amount in | Percent by | (5x initial | |
| Nectar | teaspoons | volume | ratio) | Process |
| Fructose | 1.00 | 41.67% | 5.00 | |
| solution* | | | | |
| Sucrose | 0.50 | 20.83% | 2.50 | |
| solution* | | | | |
| Glucose | 0.50 | 20.83% | 2.50 | |
| solution* | | | | |
| Artemisia Annua | 0.40 | 16.67% | 2.00 | |
| Total | 2.40 | 100.00% | | Strained |
| | | | | Filtered |

TABLE 34

| | | | Amount used | |
|---|---|---|---|---|
| | Amount in | Percent by | (5x initial | |
| Nectar | teaspoons | volume | ratio) | Process |
| Fructose | 1.50 | 72.82% | 7.50 | |
| solution* | | | | |
| Sucrose | 0.25 | 12.14% | 1.25 | |
| solution* | | | | |
| Glucose | 0.25 | 12.14% | 1.25 | |
| solution* | | | | |
| Artemisia Annua | 0.06 | 2.91% | 0.30 | |
| Total | 2.06 | 100.00% | | Strained |
| | | | | Filtered |

TABLE 35

| | | | Amount used | |
|---|---|---|---|---|
| | Amount in | Percent by | (5x initial | |
| Nectar | teaspoons | volume | ratio) | Process |
| Fructose | 1.00 | 48.54% | 5.00 | |
| solution* | | | | |
| Sucrose | 0.50 | 24.27% | 2.50 | |
| solution* | | | | |
| Glucose | 0.50 | 24.27% | 2.50 | |
| solution* | | | | |
| Artemisia Afra | 0.06 | 2.91% | 0.30 | |
| Total | 2.06 | 100.00% | | Strained |
| | | | | Filtered |

TABLE 36

| | | | Amount used | |
|---|---|---|---|---|
| | Amount in | Percent by | (5x initial | |
| Nectar | teaspoons | volume | ratio) | Process |
| Fructose | 1.00 | 47.62% | 5.00 | |
| solution* | | | | |
| Sucrose | 0.50 | 23.81% | 2.50 | |
| solution' | | | | |
| Glucose | 0.50 | 23.81% | 2.50 | |
| solution* | | | | |
| Artemisia Afra | 0.10 | 4.76% | 0.50 | |
| Total | 2.10 | 100.00% | | Strained |
| | | | | Filtered |

TABLE 37

| | | | Amount used | |
|---|---|---|---|---|
| | Amount in | Percent by | (5x initial | |
| Nectar | teaspoons | volume | ratio) | Process |
| Sucrose | 1.00 | 45.45% | 5.00 | |
| Fructose | 0.50 | 22.73% | 2.50 | |
| Glucose | 0.50 | 22.73% | 2.50 | |
| Artemisia Afra | 0.20 | 9.09% | 1.00 | |
| Total | 2.20 | 100.00% | | Strained |
| | | | | Filtered |

TABLE 38

| | | | Amount used | |
|---|---|---|---|---|
| | Amount in | Percent by | (5x initial | |
| Nectar | teaspoons | volume | ratio) | Process |
| Fructose | 1.00 | 41.67% | 5.00 | |
| solution* | | | | |
| Sucrose | 0.50 | 20.83% | 2.50 | |
| solution* | | | | |
| Glucose | 0.50 | 20.83% | 2.50 | |
| solution* | | | | |
| Artemisia Afra | 0.40 | 16.67% | 2.00 | |
| Total | 2.40 | 100.00% | | Strained |
| | | | | Filtered |

TABLE 39

| | | | Amount used | |
|---|---|---|---|---|
| | Amount in | Percent by | (5x initial | |
| Nectar | teaspoons | volume | ratio) | Process |
| Fructose | 1.50 | 72.82% | 7.50 | |
| solution* | | | | |
| Sucrose | 0.25 | 12.14% | 1.25 | |
| solution* | | | | |
| Glucose | 0.25 | 12.14% | 1.25 | |
| solution* | | | | |
| Artemisia Afra | 0.06 | 2.91% | 0.30 | |
| Total | 2.06 | 100.00% | | Strained |
| | | | | Filtered |

TABLE 40

| | | | Amount used | |
|---|---|---|---|---|
| | Amount in | Percent by | (5x initial | |
| Nectar | teaspoons | volume | ratio) | Process |
| Fructose | 1.50 | 74.26% | 7.50 | |
| solution* | | | | |
| Sucrose | 0.25 | 12.38% | 1.25 | |
| solution* | | | | |
| Glucose | 0.25 | 12.38% | 1.25 | |
| solution* | | | | |
| Artemisia Annua | 0.02 | 0.99% | 0.10 | |
| Total | 2.02 | 100.00% | | Strained |
| | | | | Filtered |

TABLE 41

| | | | Amount used | |
|---|---|---|---|---|
| | Amount in | Percent by | (5x initial | |
| Nectar | teaspoons | volume | ratio) | Process |
| Fructose | 1.50 | 74.26% | 7.50 | |
| solution* | | | | |
| Sucrose | 0.25 | 12.38% | 1.25 | |
| solution* | | | | |
| Glucose | 0.25 | 12.38% | 1.25 | |
| solution* | | | | |
| Artemisia Afra | 0.02 | 0.99% | 0.10 | |
| Total | 2.02 | 100.00% | | Strained |
| | | | | Filtered |

TABLE 42

| | Amount in | Percent by | Total | |
|---|---|---|---|---|
| Nectar | teaspoons | volume | used | Process |
| Canola oil | 1.27 | 37.41% | | |
| Fructose | 1.00 | 29.46% | | |
| solution* | | | | |
| Sucrose | 0.50 | 14.73% | | |
| solution* | | | | |
| Glucose | 0.50 | 14.73% | | |
| solution* | | | | |
| Artemisia Annua | 0.13 | 3.68% | | |
| Total | 3.40 | 100.00% | 1 | 15 minutes in |
| | | | TBSP | centrifuge then |
| | | | | strained and filtered |

**Canola oil 7.50 ml

TABLE 43

| | Amount in | Percent by | Total | |
|---|---|---|---|---|
| Nectar | teaspoons | volume | used | Process |
| Canola oil** | 1.27 | 37.41% | | |
| Fructose | 1.00 | 29.46% | | |
| solution* | | | | |
| Sucrose | 0.50 | 14.73% | | |
| solution* | | | | |
| Glucose | 0.50 | 14.73% | | |
| solution* | | | | |
| Artemisia Afra | 0.13 | 3.68% | | |
| Total | 3.40 | 100.00% | 1 | 15 minutes in |
| | | | TBSP | centrifuge then |
| | | | | strained and filtered |

**Canola oil 7.50 ml

What is claimed is:

1. A patty comprising:
a protein;
one or more types of sugars;
a lipid; and
Artemisia.

2. The patty of claim 1,
wherein the Artemisia is Artemisia annua.

3. The patty of claim 1,
wherein the Artemisia is afra.

4. A nectar comprising:
one or more types of sugars; and
Artemisia.

5. The nectar of claim 4,
wherein the Artemisia is annua.

6. The nectar of claim 4,
wherein the Artemisia is afra.

7. A method comprising:
heating water;
dissolving one or more types of sugars in the water;
cooling the water/syrup mixture;
adding Artemisia to the water/syrup mixture to form an infused water/syrup mixture; and
after a predetermined period of time, removing the Artemisia from the infused water/syrup mixture.

8. The method of claim 7, further comprising:
mixing protein and lipids into the infused water/syrup mixture; and
forming a patty.

9. The method of claim 7, further comprising:
straining and filtering solids from the infused water/syrup mixture.
